(19) United States
(12) Patent Application Publication
(10) Pub. No.: US 2016/0106243 A1
(43) Pub. Date: Apr. 21, 2016

(54) AROMA ENHANCED UTENSILS, SYSTEMS, KITS AND METHODS

(71) Applicant: Saveurs Molecule-R Inc., Montreal (CA)

(72) Inventors: Jonathan Coutu, Montreal (CA); Jerome de Champlain, Montreal (CA)

(21) Appl. No.: 14/514,525

(22) Filed: Oct. 15, 2014

(51) Int. Cl. A47G 21/02; A45C 11/00; A61L 9/12

(52) U.S. Cl. CPC A47G 21/023; A61L 9/12; A45C 11/00

(57) ABSTRACT

An aroma-enhanced eating system can include an absorbent pad, an aromatic liquid for being at least partially absorbed into the absorbent pad, and a utensil that has an aroma-diffusing section with a retention mechanism for removably retaining the absorbent pad. The aroma-diffusing section can be configured to engage the absorbent pad to provide an exposed surface area of the absorbent pad and a covered area of the absorbent pad, the exposed area of the absorbent pad being configured to enable a portion of the aromatic liquid to vaporize and diffuse in gaseous phase that is above a detection threshold to a user during use of the utensil for consumption of food. An aroma-enhanced utensil, a method of providing an aroma during consumption of food, and an aroma-enhancement kit are also described.

### REFERENCE TO RELATED APPLCATIONS

This application claims priority benefit of U.S. Provisional application 61/891,080, filed Oct. 15, 2013, the entire contents of which are hereby incorporated by reference.

### TECHNICAL FIELD

The technical field generally relates to aroma-enhancement, utensils and dining; and more particularly to techniques for enhanced aromatic experiences during food consumption.

### BACKGROUND

During certain activities, such as food ingestion, it can be desirable to experience various flavors and aromas, which can be through the tongue, orthonasal olfaction and/or retronasal olfaction for example. Experiencing flavor and aroma combinations can be particularly pleasant, interesting and educational. Of course, food flavors are typically experienced by using a utensil to engage food for consumption by a diner. However, typical utensils are often limited in the flavors and aromas that can be provided.

There are a variety of challenges associated with efficient and effective delivery of aromatic experiences to a user.

### SUMMARY

In some implementations, there is provided an aroma-enhanced eating system, comprising:
an absorbent pad;
an aromatic liquid for being at least partially absorbed into the absorbent pad; and
a utensil comprising:
a main body comprising:
   a handle portion; and
   a food-engaging portion; and
an aroma-diffusing section connected to the main body, comprising:
   a retention mechanism for removably retaining the absorbent pad having the aromatic liquid absorbable therein;
   the aroma-diffusing section being configured to engage the absorbent pad to provide an exposed surface area of the absorbent pad and a covered area of the absorbent pad, the exposed area of the absorbent pad being configured to enable a portion of the aromatic liquid to vaporize and diffuse in gaseous phase that is above a detection threshold to a user during use of the utensil for consumption of food.

In some implementations, the retention mechanism comprises:
a connection member connected to the main body and extending outwardly therefrom; and
a clamping member connected to a distal end of the connection member and extending in spaced relation with respect to an opposed surface of the main body, thereby defining there-between a region for receiving the absorbent pad.

In some implementations, the region is slot-shaped and the absorbent pad has a thickness that generally corresponds to a thickness of the slot-shaped region, such that the absorbent pad occupies substantially the entire thickness of the region.

In some implementations, the clamping member comprises a first opening configured to expose a portion of a first surface of the absorbent pad and providing part of the exposed surface area and

In some implementations, the main body comprises a second opening configured to expose a portion of a second surface of the absorbent pad providing part of the exposed surface area.

In some implementations, the main body comprises a neck portion in between the handle portion and the food-engaging portion, and the retention mechanism is connected to the neck portion.

In some implementations, the exposed surface area of the absorbent pad is at least 100 mm². In some implementations, the exposed surface area of the absorbent pad is between 200 mm² and 300 mm². In some implementations, the covered surface area of the absorbent pad is between 100 mm² and 150 mm².

In some implementations, the covered surface area of the absorbent pad is compressed to provide retention of the absorbent pad.

In some implementations, the absorbent pad is composed of un-sized paper material. In some implementations, the absorbent pad is composed of blotting paper. In some implementations, the absorbent pad is composed of multiple layers of blotting paper.

In some implementations, the absorbent pad has an absorbency rate sufficiently high to absorb a drop of the aromatic liquid upon contact and thereby inhibiting pearling or beading of the aromatic liquid.

In some implementations, the absorbent pad has an absorbency so as to prevent release of the aromatic liquid absorbed therein.

In some implementations, there is provided an aroma-enhanced utensil, comprising:
a main body comprising:
   a handle portion; and
   a food-engaging portion; and
an aroma-diffusing section connected to the main body, comprising:
   a retention mechanism for removably retaining an absorbent pad having an aromatic liquid absorbable therein;
   the aroma-diffusing section being configured to engage the absorbent pad to provide an exposed surface area of the absorbent pad and a covered area of the absorbent pad, the exposed area of the absorbent pad being configured to enable a portion of the aromatic liquid to vaporize and diffuse in gaseous phase that is above a detection threshold to a user during use of the utensil for consumption of food.

In some implementations, there is provided a method of providing an aroma during consumption of food, comprising: providing a utensil; removably retaining an absorbent pad to the utensil; and absorbing an aromatic liquid into the absorbent pad such that a portion of the aromatic liquid vaporizes and diffuses in gaseous phase above a detection threshold to a user during use of the utensil for consumption of food.

In some implementations, there is provided an aroma-enhancement kit, comprising: a set of the utensils each comprising at least one retention mechanism; a set of containers containing different aromatic liquids; and a set of the absorbent pads each being removably retainable the retention mechanism of a corresponding one of the utensils.

In some implementations, the kit further comprises: at least one dropper for transferring drops of the aromatic liquids from the containers to the absorbent pads; and a tray comprising designated supports or indentations for respectively receiving the utensils, the containers and the at least one dropper.

In some implementations, the indentation of the tray correspond to the shape and size of each respective utensil, container and dropper to provide lateral retention on the tray.

### BRIEF DESCRIPTION OF DRAWINGS

FIG. 1 is a front view of a utensil.

FIG. 2 is a back view of a utensil with a retention mechanism in an open position.

FIG. 3 is a back view of a utensil with a retention mechanism in a closed position.

FIG. 4 is a side view of a utensil with a retention mechanism in a closed position.

FIG. 5 is a side partially transparent view of part of a utensil.

FIG. 6 is a top plan view of part of a utensil.

FIG. 7 is a side partially transparent view of part of a utensil.

FIG. 8 is a side partially transparent view of part of a utensil.

FIGS. 9A to 9D are schematics of openings that can be in part of a utensil.

FIGS. 10A to 10C are side cut views of part of a utensil showing an opening.

FIGS. 11 A to 11 C are perspective views of absorbent pads.

FIG. 12 is a side view of a dropper.

FIG. 13 is a top perspective view of a serving plate.

FIG. 14 is a perspective view of a utensil.

FIG. 15 is a side view of a pen including an aroma diffusing section.

### DETAILLED DESCRIPTION

Techniques described herein enable aromas to be delivered to a user. In some implementations, the aroma is delivered to a diner during consumption of food using a utensil on which an absorbent pad containing aromatic liquid is retained. Part of the absorbent pad exposed so as to enable vaporization and diffusion of the aromatic substance in gaseous phase aroma for detection by the diner. The absorbent pads can be removable and replaceable in order to provide a variety of aromatic experiences.

During food ingestion, the flavors that are recognized by a diner are most often associated with taste and are detected by the tongue. The tongue includes taste buds that are capable of distinguishing five common tastes: sweet, sour, salty, sour and umami. These five tastes provide limited subtlety and cannot easily distinguish the difference in flavors between some relatively different items (e.g. a red cherry and a strawberry). Additional complexity that is experienced during food ingestion is provided by olfaction and nerves present in the nose that can differentiate thousands of flavors. Flavours are appreciated via orthonasal olfaction that occurs as an individual inhales, and also via retronasal olfaction when an individual exhales. The odors of the food, first present in the oral cavity, travel backward to reach the throat which is connected to the nasal cavity and its olfactory receptors. It is during the exhalation phase that retronasal olfaction occurs and further complexity of flavors may be experienced.

In some implementations, the utensil provides users with a very distinct food tasting and ingestion experience, facilitating the experience of different flavors as during inhalation the diverse pairing of foods and aromas. The combination of taste, retronasal olfaction and orthonasal olfaction can produce a very rich experience.

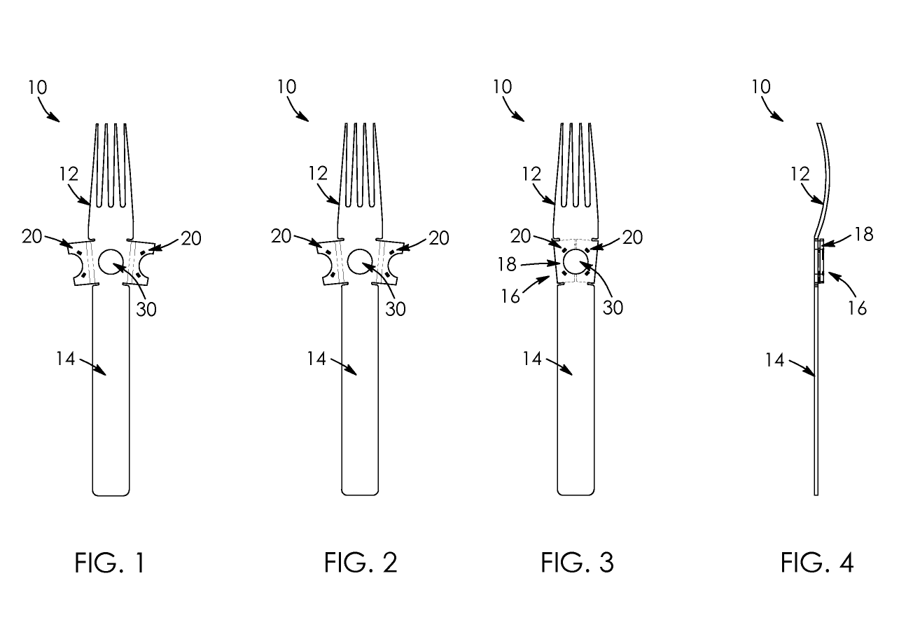

Referring to FIGS. 1 to 4, in some implementations a utensil 10 is provided and has a food-engaging or food-contacting section 12, which in this case is illustrated as the tines of a fork. However, in other implementations, the utensil can be a spoon, a knife, chopsticks, or another type of utensil. The utensil 10 also includes a handle portion 14, for handling by a user.

Referring now to FIGS. 3 and 4, the utensil 10 also includes an aroma-diffusing section 16. The aroma-diffusing section 16 can include a retention mechanism 18 configured for removably retaining an absorbent pad (not illustrated here). In FIGS. 3 and 4, the retention mechanism 18 is formed by two opposed wings 20 that extend from opposed lateral sides of the utensil 10 and are folded inward such that the distal edges of the wings come into proximate relation or are aligned and contacted. FIGS. 1 and 2 illustrate the wings 20 in an open position, and during manufacturing the wings 20 would be folded together as shown in FIGS. 3 and 4. In some implementations, the retention mechanism is formed of relatively rigid elements that do not significantly bend or change configuration, although in some alternative scenarios the retention can include moving parts.

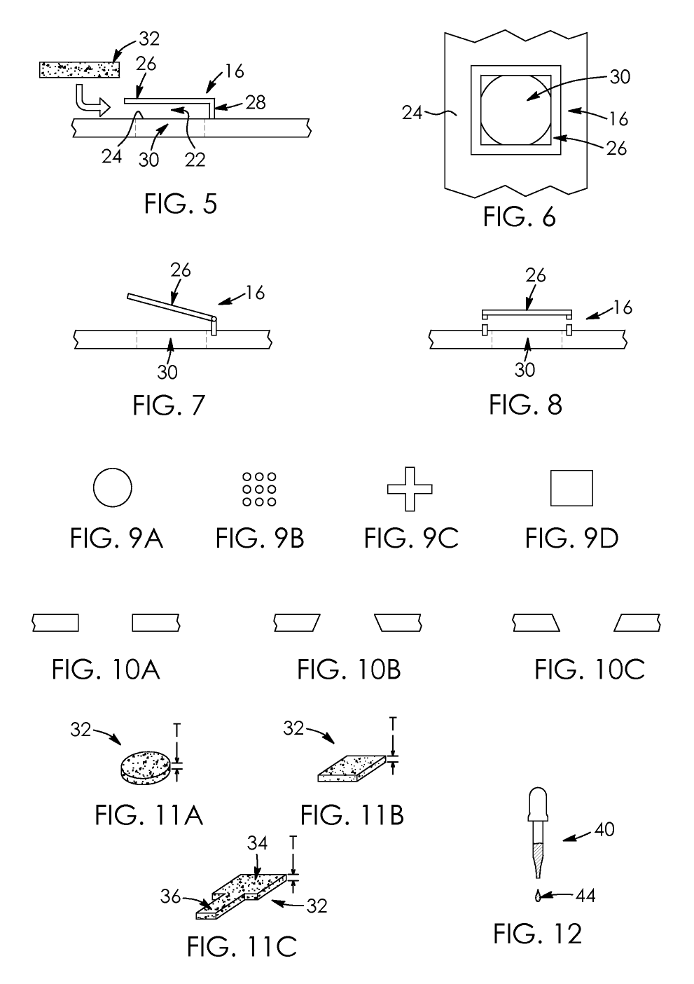

Referring now to FIG. 5, the retention mechanism 16 can be configured in order to define a region 22, such as a slot, which can be defined in between an upper surface 24 of the fork and a clamp member 26 that is spaced apart from the upper surface 24. The clamp member 26 can be connected to the rest of the utensil via a connection member 28, which can be welded, glued or otherwise attached.

Referring still to FIG. 5, in some implementations, the utensil also includes an opening 30 through the main body of the utensil. The region 22 and the opening 30 are sized and configured such that an absorbent pad 32 can be received and retained in the region 22 and a portion of the absorbent pad 32 is exposed to the atmosphere through the opening 30.

Turning to FIGS. 3, 4 and 6, in some implementations, the retention mechanism is also shaped to define a second opening through which the absorbent pad is exposed on one side and the opening 30 in the main body of the utensil enables the absorbent pad to be exposed on the opposed side. Such tow-sided exposure can further facilitate vaporization of aromatic liquids that are absorbed into the absorbent pad so that the vaporised aromatic substances can be detected and appreciated by a user.

Referring to FIGS. 5 and 6, the retention mechanism 16 can include two bars as the connection member 28, extending outward from the surface of the utensil, for example at a right angle, to help define the thickness of the region into which the absorbent pad will be inserted. The retention member 16 can also include a square-shaped member as the clamp member 26, with a central square opening. The clamp member 26 can be configured in generally parallel relation to the upper surface of the utensil, thereby defining the region that is generally uniform in thickness over the area. It should also be noted that the connection member 28 and the clamp member 26 can be provided as a generally one-piece structure. In addition, the retention mechanism 16 can be constructed to have resiliency such that the clamp member 26 can be moved upward to facilitate insertion of the absorbent pad and then springs back in order to clamp down the absorbent member against the opposed surface of the utensil.

Referring now to FIGS. 7 and 8, the retention mechanism 16 can have various other constructions, which can include a clamp member 26 connected to the utensil body by a hinge, for clamping the absorbent pad (as in FIG. 7) or a detachable clamp member 26 that can be removed and then clipped or otherwise attached to the utensil body to clamp the absorbent pad.

Turning now to FIGS. 9A to 9D, the openings provided on the clamp member and/or in the main body of the utensil can have various shapes and sizes, some of which are illustrated. The two openings can have similar or different sizes and may be aligned with each other or not.

Referring to FIGS. 10A to 10C, the openings provided on the clamp member and/or in the main body of the utensil can also be defined by various side wall orientations, some of which are illustrated. In some implementations, at least one of the openings is sized to be sufficiently large such that drops of the aromatic liquid can be discharged there-through to contact the absorbent pad while inhibiting or avoiding contact of the liquid with the utensil.

Referring to FIGS. 11 A to 11 C, the absorbent pads 32 have a size, shape and configuration to enable retention by the retention mechanism. In some implementations, the absorbent pads 32 are provided to be removably insertable into the region and to be clamped by the retention mechanism. The absorbent pads 32 can have a variety of shapes, including circular or square or more complex shapes. FIG. 11 C illustrates an implementation in which the absorbent pad 32 has a main portion 34 that can be accommodated within the region and receives the aromatic liquid, and a stub portion 36 that extends out of the region and facilitates removal by a user by pulling on it.

In some implementations, the absorbent pads are composed of an absorbent material that provides desired liquid absorption and vaporization properties. The absorbent material facilitates rapid liquid absorption past the outer surface so as to form a liquid-impregnated region of the absorbent pad, thereby reducing pearling or beading of the liquid on the outer surface of the absorbent pad. However, the absorbent material and the sizing of the absorbent pad are such that the aromatic liquid absorbed into the pad can have a desired vaporization rate sufficient to allow diffusion of the gaseous phase aromatic substance for detection by a user. Thus, the absorbent pads should have liquid absorption properties for rapid absorption while enabling sufficient aromatic liquid to be exposed at the outer surface of the pad to facilitate vaporization.

In some implementations, the absorbent pads are composed of an absorbent material including cellulosic-based and/or polysaccharide-based materials. The absorbent pads can include multiple layers or sheets of such materials. For example, the absorbent pads can be composed of blotting paper, which can include from 8 to 16 layers. In some implementations, the absorbent pads are composed on un-sized paper. In some implementations, the absorbent pads have an absorbency capacity for retaining at least one or a few drops of aromatic liquid while inhibiting liquid leakage, an absorbance rate sufficiently high to promote rapid absorbance of one or a few drops of aromatic liquid to inhibit beading or pearling on the pad surface, and swelling properties such that the volumetric increase of the pad upon addition of the aromatic liquid is relatively low, for example to avoid significant difficultly in removing the pad from the retention member after use. In some implementations, the absorbent pads are composed of an absorbent material for absorbing certain types of aromatic liquids, such as aqueous liquids, organic liquids, oil-in-water emulsions, water-in-oil emulsions, and various other mixtures that may have desired aromatic properties. In some implementations, the pad has absorbency such that a drop is absorbed within 1 second after contact.

In some implementations, the absorbent pad has a total surface area (A_{T}) including its opposed surfaces and side edge, and the pad and the retention mechanism are sized and configured to cooperate together so that the pad has a total exposed surface area (A_{E}) of between 100 mm² and 400 mm², 150 mm² and 350 mm², or 200 mm² and 300 mm², and the pad also has a total compressed surface area (A_{C}) that engages the clamping member or part of the body of the utensil of between 50 mm²and 200 mm², 75 mm² and 175 mm², or 100 mm² and 150 mm². In some implementations, the side edge of the absorbent pad is completely exposed while parts of the opposed surfaces are exposed while other parts are compressed in between the clamping member and the main body of the utensil. The A_{E}/A_{C ratio} can be provided to facilitate secure yet removable retention of the pad as well as vaporization of sufficient aromatic compounds from the pad. In some implementations, the AE and the pad volume and absorbency properties can be configured to provide sufficient vaporization of the aromatic compound such that the aromatic vapours are present above a corresponding detection threshold when 5 or 10 cm or less away, or above a corresponding identification threshold when about 5 or 10 cm away.

In some implementations, the region defined between the clamping member and the body can be slightly smaller than the thickness (T) of the pad and the pad is compressible for insertion. In this way, insertion of the absorbent pad includes compressing the pad to reduce its thickness and provide the compressive force for retention. This may be used in particular when the retention member has a high rigidity and lower resiliency to provide the biasing force for retention. In addition, the pad can have a size and configuration such that at least a portion of the pad extends beyond the clamping member and is engageable by a user to facilitate removal by pushing or pulling. For example, the clamping member can engage a surface of the pad so as to define exposed outer segments (e.g., circle segments when the pad is disk shaped), so that a user can easily engage one or more of the exposed segments to slide the pad out of the region for disposal, replacement, cleaning, and so on. In some implementations, there are four segments exposed in four quadrants of the pad, further facilitating removal options from and to various directions. Other removal techniques can include moving the clamping member away from the pad, for example.

In some implementations, the absorbent pads are composed of inedible material such as cellulose-based paper materials. In alternative scenarios, the absorbent pads can be made from edible materials, which can include polysaccharides (e.g., starch), modified polysaccharides, or other

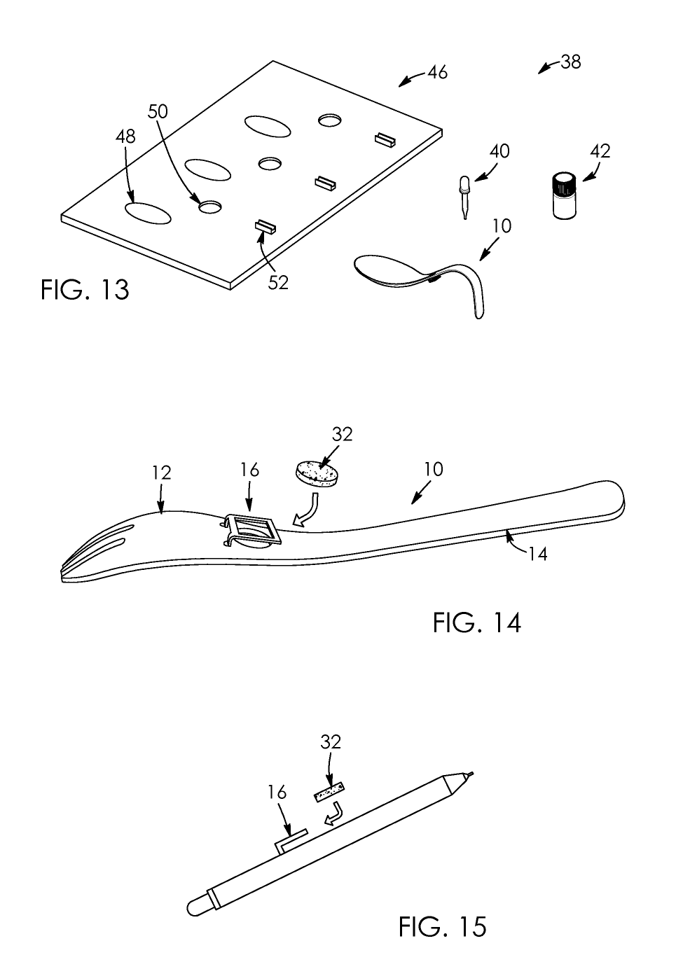

Referring to FIG. 13, an aroma enhancement kit 38 can be provided including a set of the utensils 10 (here illustrated as a spoon), a set of droppers 40, a set of vials 42 each containing an aromatic liquid 44, and a tray or plate 46 that has a number of locations for placing the utensils, droppers and vials. The tray 46 can include utensil indentations 48 for receiving corresponding utensils, vial indentations 50 for receiving corresponding vials, and dropper supports 52 for receiving corresponding droppers. The items can thus be arranged and aesthetically presented, for example at a restaurant or diner party.

It should be noted that the aroma diffusing section 16 can have various other constructions that includes a mechanism for removably retaining absorbent pads while allowing at least part of the absorbent pad that has received an aromatic liquid is exposed to the atmosphere to enable vaporization of the aromatic material. For example, the aroma diffusing section may include a recess or groove within the main body of the utensil, in which the absorbent pad may be placed and held into position. In addition, there may be one or more of the diffusing sections provided along the utensil and can be on any part of the handle or neck of the utensil for example. The retention member can also be provided on the back of the utensil, as illustrated, or on the side or front.

In terms of the aromatic liquids that can impregnate the absorbent pads, various aromatic materials and substances can be used in combination with carriers (e.g., water or oil) and other compounds. The aromatic liquid that is provided can also be edible. In some implementations, the aroma can be obtained from concentrated aromatic extracts such as oils (e.g., essential oils), tinctures, solid extracts obtained from original material, or even the pure aromatic molecule obtained from extraction or chemical synthesis. In some alternative scenarios, the aroma may come from a piece or fragment from the original material (e.g., citrus fruit peel, wood chips, spices, any suipadle portion of the spice material such as the leaves, stem, root, and so on. The aroma can come from a single source, or a mixture or sources in order to obtain more complex combinations. Examples of aromas include but are not limited to orange, apple, grapefruit, pineapple, strawberry, raspberry, cranberry, lime, lemon, grape, peach, any other fruit flavoring, vanilla, chocolate, caramel, maple or any other combination thereof.

In operation, the user can select combination of food item and aroma, provide one or more drops of the corresponding aromatic liquid onto a surface of the absorbent pad (e.g., prior to or after engagement by the retention member), and then proceed with using the utensil for eating the food item while the aroma vapours are released. The aroma vapours can be appreciated before, during and/or after eating. This provides the opportunity to make interesting pairings, for example inhaling the smell of basil before eating a tomato salad, or vanilla before eating a banana cake, or more unusual combinations.

In some alternative scenarios, an aroma diffusing mechanism as described herein can be provided on other hand-held items or tools, such as pens, pencils, other writing instruments, key rings, glasses or cups, watches, rings, jewelry, or other items, for aroma therapy purposes without the involvement of food associated with that tool or its use. In such scenarios, there may be provided a kit that includes pads, a retention mechanism and an aromatic liquid for use in association with the item. The retention mechanism can be provided as an integral part of the item (e.g., permanently attached to the rest of the item) or may be provided separately from the item and provided with a connection mechanism (e.g., strap, clip, snap-on device, Velcro™, etc.) for connecting the retention mechanism to the item. FIG. 15 illustrates this alternative scenario for a pen. The other figures relate to when the item or tool is a utensil.

Thus, in some scenarios, an aroma-enhanced tool is provided, comprising:
a main body comprising:
   a handle portion;
   a functional tool end portion;
an aroma-diffusing section connected to the main body, comprising:
   a retention mechanism for removably retaining an absorbent pad having an aromatic liquid absorbable therein, the aroma-diffusing section being configured to expose a portion of the absorbent pad to atmosphere to enable a portion of the aromatic liquid to vaporize and diffuse in gaseous phase.

The aroma-diffusing section provided with respect to the tool can have any one or more of the features described or illustrated herein for the utensil.

According to testing, the absorbent tabs were composed of a multi-layer blotting paper material that provided effective absorption properties, including rapid absorption rate, liquid retention, and aroma vaporization above detection and identification thresholds. Test pads that were composed of a single layer of lower absorbent material resulted in the drops beading at surface and/or degradation of structural integrity.

1. An aroma-enhanced eating system, comprising:
an absorbent pad;
an aromatic liquid for being at least partially absorbed into the absorbent pad; and
a utensil comprising:
a main body comprising:
a handle portion; and
a food-engaging portion; and
an aroma-diffusing section connected to the main body, comprising:
a retention mechanism for removably retaining the absorbent pad having the aromatic liquid absorbable therein;
the aroma-diffusing section being configured to engage the absorbent pad to provide an exposed surface area of the absorbent pad and a covered area of the absorbent pad, the exposed area of the absorbent pad being configured to enable a portion of the aromatic liquid to vaporize and diffuse in gaseous phase that is above a detection threshold to a user during use of the utensil for consumption of food.

2. The aroma-enhanced eating system of claim 1, wherein the retention mechanism comprises:
a connection member connected to the main body and extending outwardly therefrom; and
a clamping member connected to a distal end of the connection member and extending in spaced relation with respect to an opposed surface of the main body, thereby defining there-between a region for receiving the absorbent pad.

3. The aroma-enhanced eating system of claim 2, wherein the region is slot-shaped and the absorbent pad has a thickness that generally corresponds to a thickness of the slot-shaped region, such that the absorbent pad occupies substantially the entire thickness of the region.

4. The aroma-enhanced eating system of claim 2, wherein the clamping member comprises a first opening configured to expose a portion of a first surface of the absorbent pad and providing part of the exposed surface area and

[symbol]

5. The aroma-enhanced eating system of claim 4, wherein the main body comprises a second opening configured to expose a portion of a second surface of the absorbent pad providing part of the exposed surface area.

6. The aroma-enhanced eating system of claim 2, wherein the main body comprises a neck portion in between the handle portion and the food-engaging portion, and the retention mechanism is connected to the neck portion.

7. The aroma-enhanced eating system of claim 1, wherein the exposed surface area of the absorbent pad is at least 100 mm2.

8. The aroma-enhanced eating system of claim 1, wherein the exposed surface area of the absorbent pad is between 200 mm² and 300 mm².

9. The aroma-enhanced eating system of claim 8, wherein the covered surface area of the absorbent pad is between 100 mm2 and 150 mm2.

10. The aroma-enhanced eating system of claim 9, wherein the covered surface area of the absorbent pad is compressed to provide retention of the absorbent pad.

11. The aroma-enhanced eating system of claim 10, wherein the absorbent pad is composed of un-sized paper material.

12. The aroma-enhanced eating system of claim 11, wherein the absorbent pad is composed of blotting paper.

13. The aroma-enhanced eating system of claim 12, wherein the absorbent pad is composed of multiple layers of blotting paper.

14. The aroma-enhanced eating system of claim 1, wherein the absorbent pad has an absorbency rate sufficiently high to absorb a drop of the aromatic liquid upon contact and thereby inhibiting pearling or beading of the aromatic liquid.

15. The aroma-enhanced eating system of claim 1, wherein the absorbent pad has an absorbency so as to prevent release of the aromatic liquid absorbed therein.

16. An aroma-enhanced utensil, comprising:
a main body comprising:
a handle portion; and
a food-engaging portion; and
an aroma-diffusing section connected to the main body, comprising:
a retention mechanism for removably retaining an absorbent pad having an aromatic liquid absorbable therein;
the aroma-diffusing section being configured to engage the absorbent pad to provide an exposed surface area of the absorbent pad and a covered area of the absorbent pad, the exposed area of the absorbent pad being configured to enable a portion of the aromatic liquid to vaporize and diffuse in gaseous phase that is above a detection threshold to a user during use of the utensil for consumption of food.

17. A method of providing an aroma during consumption of food, comprising:
providing a utensil;
removably retaining an absorbent pad to the utensil; and
absorbing an aromatic liquid into the absorbent pad such that a portion of the aromatic liquid vaporizes and diffuses in gaseous phase above a detection threshold to a user during use of the utensil for consumption of food.

18. A aroma-enhancement kit, comprising:
a set of the utensils each comprising at least one retention mechanism;
a set of containers containing different aromatic liquids; and
a set of the absorbent pads each being removably retainable the retention mechanism of a corresponding one of the utensils.

19. The aroma-enhancement kit of claim 18, further comprising:
at least one dropper for transferring drops of the aromatic liquids from the containers to the absorbent pads; and
a tray comprising designated supports or indentations for respectively receiving the utensils, the containers and the at least one dropper.

20. The aroma-enhancement kit of claim 19, wherein the indentation of the tray correspond to the shape and size of each respective utensil, container and dropper to provide lateral retention on the tray.
